(19) United States
(12) Patent Application Publication
(10) Pub. No.: US 2026/0165787 A1
(43) Pub. Date: Jun. 18, 2026

(54) MEDICAL PATIENT SIMULATION TRAINING SYSTEM

(71) Applicant: Board of Trustees of Southern Illinois University, Springfield, IL (US)

(72) Inventors: Richard Selinfreund, Springfield, IL (US); Pinckney Benedict, Springfield, IL (US); Akhila Kambhatla, Springfield, IL (US)

(21) Appl. No.: 19/359,217

(22) Filed: Oct. 15, 2025

Related U.S. Application Data
(60) Provisional application No. 63/710,941, filed on Oct. 23, 2024.

(51) Int. Cl. A61B 34/10; G09B 5/02; G16H 50/20

(52) U.S. Cl. CPC A61B 34/10; G09B 5/02; G16H 50/20

(57) ABSTRACT

A medical patient simulation training system includes a display interface for a trainee using the training system. The system also includes a trained artificial intelligence agent. The agent is trained on a codebook for disease-specific interactions. Real-time software quantifies the use of codebook words and phrases, assesses diagnostic accuracy and emotional engagement of the trainee during a simulation training session. A system of the invention can include an autonomous AI platform featuring visually simulated patients trained to provide voice-to-voice communication with natural intonations and to interact and respond to a trainee. It can include a local language model trained on specialty language codebooks tailored for disease-specific a simulation interaction with a trainee. The real-time software can assess diagnostic accuracy and emotional engagement of the trainee during the simulation interaction.

### PRIORITY CLAIM AND REFERENCE TO RELATED APPLICATION

The application claims priority under 35 U.S.C. § 119 from prior U.S. provisional application Ser. No. 63/710,941, which was filed Oct. 23, 2024. All applications mentioned in this paragraph are incorporated by reference.

### FIELD

Fields of the invention include medical simulation systems and medical training tools and systems.

### BACKGROUND

Simulation systems including haptic feedback are provided for various professions, including pilot training systems and surgical simulation systems. Such simulation systems are critical to the development of professional skills.

Medical practice requires more than physical surgical skills to provide the most effective training for medical professionals. Diagnosis is key to the most effective medical care. Medical professionals can struggle early in their careers in obtaining patient information that is critical to providing an effective diagnosis. Critical information comes from both observation of a patient and communication with the patient, including verbal and nonverbal communication.

Traditional methods of training, such as role-playing or standardized patients, can be limited in scope and consistency. There is a need for a more scalable, consistent, and customizable approach to creating patient scenarios for medical training.

Existing medical simulations are almost exclusively designed as high-fidelity, deterministic systems. They function as digital textbooks; a medical learner follows a set path to arrive at a predictable, correct outcome. They are rigid, single-purpose tools designed for rote memorization and procedural practice. These are known as ePBLM (electronic problem based learning) systems. See, e.g., Ryan and Koschmann, “The Collaborative Learning Laboratory: A Technology-Enriched Environment to Support Problem-Based Learning,” National Educational Computing Conference 1994.

### SUMMARY

A preferred medical patient simulation training system includes a display interface for a trainee using the training system. The system also includes a trained artificial intelligence agent. The agent is trained on a codebook for disease-specific interactions. Real-time software quantifies the use of codebook words and phrases, assesses diagnostic accuracy and emotional engagement of the trainee during a simulation training session.

A preferred system of the invention can include an autonomous AI platform featuring visually simulated patients trained to provide voice-to-voice communication with natural intonations and to interact and respond to a trainee. It can include a local language model trained on specialty language codebooks tailored for disease-specific a simulation interaction with a trainee. The real-time software can assess diagnostic accuracy and emotional engagement of the trainee during the simulation interaction.

### BRIEF DESCRIPTION OF THE DRAWINGS

FIGS. 1A-1D show a preferred medical patient simulation training system.

FIG. 2 shows a mobile device with an example display during a simulated patient interaction using an app of a preferred medical patient simulation training system.

FIG. 3 shows a Venn diagram of the sentiment analysis used in preferred embodiment medical patient simulation training systems.

FIG. 4 shows a preferred medical patient simulation training system that includes a cameras positioned to capture both a trainee and a virtual patient.

### DETAILED DESCRIPTION OF THE PREFERRED EMBODIMENTS

Preferred embodiments provide a medical patient simulation training system. A preferred embodiment system includes an interface for a medical trainee that provides access to an artificial intelligence based medical patient that is generated by the system. The system monitors verbal communications between the trainee and the AI patient and includes a codebook that identifies a list of key words or phrases specific a medical specialty (i.e. medical education, emergency room training, improving physician Press Ganey scores in their clinical specialty). Preferably, each specialty includes a separate codebook. The system conducts a sentiment analysis of the AI patient in real time through measurement of codewords and phrases in the codebook.

Preferred systems provide a new approach to medical education that generates and uses Autonomous Artificial Intelligence Simulated Patients. The systems can be trained by actors portraying real-world patients and conditions, or recorded sessions of actual patients (if available with consent) patient medical histories, personality profiles, demographic group tendencies, and the causal relationship of medical conditions on different personality profiles and demographic groups.

A preferred system integrates A) an autonomous AI platform featuring visually simulated patients and voice-to-voice communication with natural intonations, creating a highly realistic learning environment. B) specialty language codebooks that trains a local language model (LLM) tailored for disease-specific interactions, ensuring accurate learning metrics. C) real-time software that quantifies the use of codebook words and phrases, assessing learners' diagnostic accuracy and emotional engagement during simulations. The synergy of these components results in a safe, real-time, specialty-matched, and cost-effective learning environment. Preferred systems can significantly improve medical education by providing immersive, adaptive, and measurable learning experiences for healthcare professionals in training.

Preferred embodiments provide a comprehensive system and method for generating artificial intelligence (AI) based medical avatars. This system integrates advanced natural language processing, image generation, and AI personality modeling to create realistic, interactive patient avatars for medical training purposes. The invention encompasses processes for data acquisition, profile generation, visual representation, and AI model integration, resulting in highly detailed and medically accurate virtual patients.

A preferred system generates AI-based medical avatars for use in healthcare training. The invention integrates medical data processing, natural language generation, image synthesis, and AI personality modeling to create detailed, interactive virtual patients. The system includes modules for data acquisition, profile generation, visual representation, and AI integration, along with processes for testing, refinement, and deployment. This invention provides a scalable and customizable approach to creating diverse patient scenarios for medical education and training.

Preferred embodiments provide an AI patient generation that:
Features visually simulated patients
Utilizes voice-to-voice communication with natural voice intonations
Provides a more realistic learning environment for medical students

Preferred embodiments provide a Matched medical learning environment that includes:
Specialty language codebooks tailored to patient interactions
Implements a local language model (LLM) specific to medical interactions
Enables disease-specific interactions and accurate learning metrics.

Preferred embodiments provide a Realtime sentiment program that:
Accurately recognizes and quantifies codebook words and phrases
Measures a medical learner's ability to increase diagnostic accuracy
Assesses sentiment connections during the simulation.

A preferred embodiment that encompasses AI patient generation, a Matched medical learning environment, and a Realtime sentiment program provide a powerful simulation tool. The components work together to create a safe, real-time, specialty-matched, and cost-effective learning environment for medical students.

Preferred embodiments include inner dialogue analysis of trainee/user and the AI agent/patient—including analysis of nonverbal signal capture, multimodal synchronization, feature extraction, and codebook-based interpretation of affective states. This preferably includes monitoring both verbal and nonverbal communication between learner and AI patient. Sentiment analysis frameworks are integrated with medical specialty codebooks to assess emotional context. Preferred systems integrate AI-driven avatars with real-time feedback for learner performance in communication and diagnostic reasoning.

Preferred embodiments of the invention will now be discussed with respect to experiments and drawings. Broader aspects of the invention will be understood by artisans in view of the general knowledge in the art and the description of the experiments that follows.

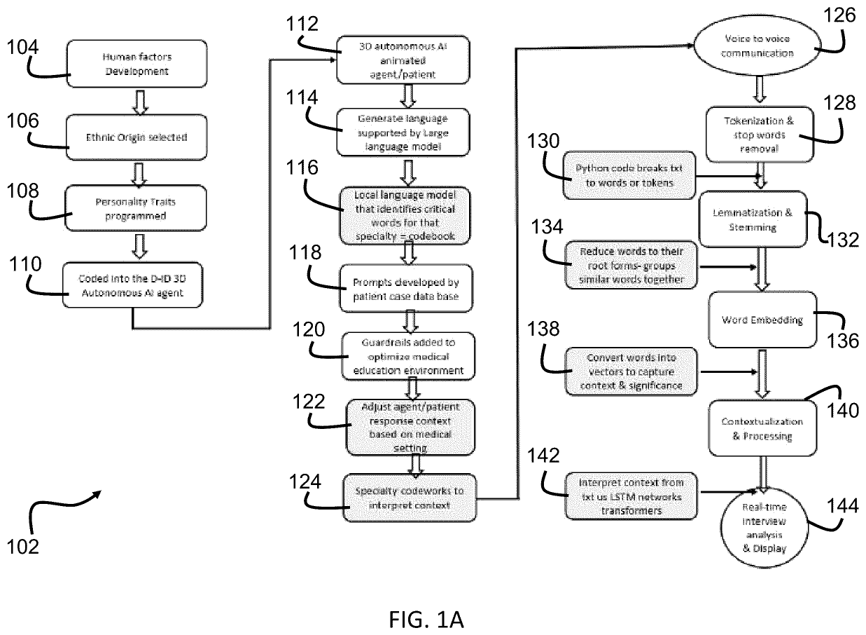

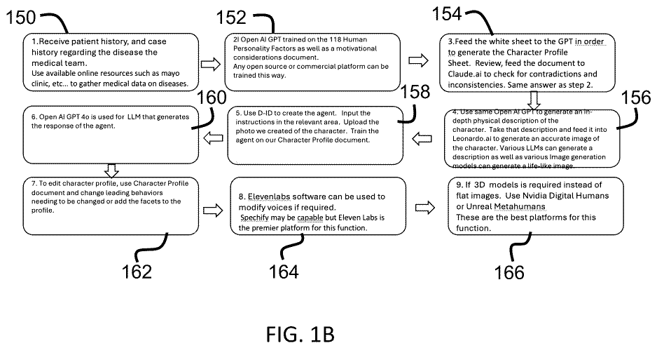

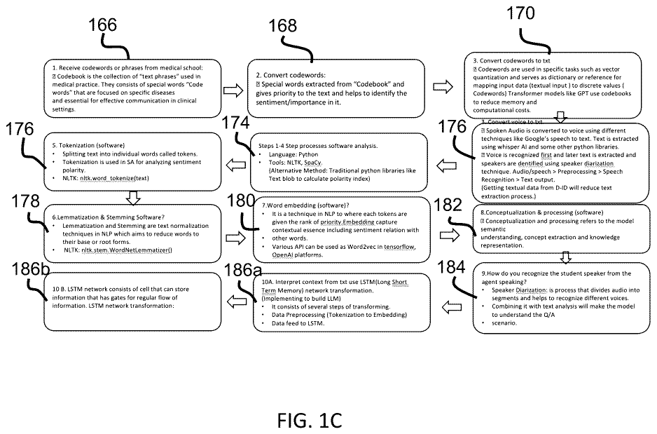

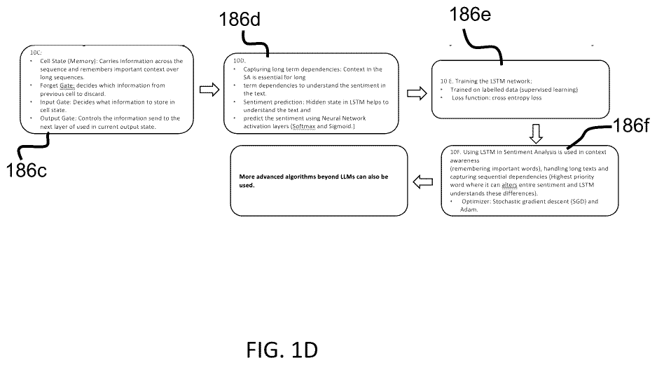

FIGS. 1A-1D show a preferred medical patient simulation training system. FIG. 1A shows steps for providing a training simulation. An initial data collection/training is used so that human factors are developed 104 based upon clinical data, which can include actors portraying real-world patients and conditions, or recorded sessions of actual patients (if available with consent) patient medical histories, personality profiles, demographic group tendencies, and the causal relationship of medical conditions on different personality profiles and demographic groups.

For a simulation, ethnic origin is selected 106, which can include demographic group tendencies for the simulation. Personality traits are also selected 108, e.g. cooperative, uncooperative, nervous, outgoing, shy, etc. Generally, any traits that can affect patient-professional interaction can be included. The selections are coded 110 for use by an autonomous AI agent, such as the DHD 3D agent. With coded selections, the agent produces 112 and animated/image version of a patient. This creates visually simulated patients, and language for the patient is created 114 by a large language model. The present training system creates a local language model 116 from a specialty language codebook. The specialty language codebooks that trains the local language model (LLM) so it is tailored for disease-specific interactions, ensuring accurate learning metrics during a simulation.

The system provides prompts 118 that define case-specific behavior for the AI-simulated patient and thereby seed the AI-simulated patient's responses during the encounter. For example, in a case of undiagnosed early-onset diabetes, the AI-simulated patient persona is configured as reluctant to be in clinic and, when queried, is prompted to disclose symptoms such as “I've been more thirsty than usual” and “I get up to urinate more at night.” These prompts drive the AI-simulated patient's answers to the medical learner's questions in a manner that is specific to the selected case. Guardrails 120 are constraint prompts that prevent inappropriate or premature disclosures and enforce scenario boundaries—for instance, the AI-simulated patient is not permitted to state or infer that the patient “has diabetes” unless the case logic authorizes that disclosure. The AI-simulated virtual patient (agent) responses are adjusted 122 according to the examination setting (e.g., clinic room, inpatient room, home visit, telehealth video), so that tone, phrasing, and environmental references remain contextually appropriate. A specialty language codebook 124 is applied to the interaction; e.g., for internal medicine, the codebook comprises curated words, question stems, and response checks that foster trust and verify understanding of the proposed treatment plan (e.g., a teach-back prompt: “Can you tell me in your own words what the plan is?”). During a learning session, the software detects these specialty codewords and phrases in real time and scores their use to produce immediate feedback to the learner. Distinct clinical specialties employ corresponding codebooks so that detection, scoring, and coaching are aligned to discipline-specific communication standards.

Simulated examination voice-to-voice communications are developed 126 to support real-time discourse between the virtual patient (agent) and the user/trainee. The audio is transcribed and the text is processed through tokenization and stop-word removal 128—the transcript is split into tokens (words/terms) and high-frequency function words (e.g., “the,” “and,” “of”) are removed so that analysis continues on content-bearing terms. Next, lemmatization and stemming 132 normalize tokens to their base forms to group related words and improve model performance (e.g., running/runs/runner→run by stemming; better→good by lemmatization using part-of-speech/context). The system then performs word embedding 136, converting normalized tokens into numerical vectors in a high-dimensional space to capture contextual essence and sentiment relations among words (e.g., embeddings learned via Word2Vec, GloVe, or transformer-based models), and may assign priority/importance weights to tokens for downstream scoring. Contextualization and processing 140 use sequence models—such as Transformers and Long Short-Term Memory (LSTM) networks—to represent long-range dependencies and utterance-level meaning beyond simple keyword matching. The preferred system 102 employs Python's OpenAI and Transformers libraries for these tasks; NLTK supports tokenization, lemmatization, and stemming; and TextBlob provides polarity and subjectivity scores utilized within the software analysis component. Once the system is ready to interface with a trainee, it provides 144 a real-time interview analysis and display that surfaces turn-by-turn feedback and quantitative metrics (e.g., latency, codebook hits, sentiment/rapport) to help students understand communication behaviors, identify strengths and areas for improvement, and aid instructor feedback.

The preferred system uses the Python NLTK library to perform tokenization 130, splitting the ASR transcript into word-level tokens, followed by stop-word removal 130 to discard high-frequency function words so downstream analysis emphasizes content-bearing terms. During lemmatization and stemming 132, tokens are normalized to their base/lemma or stem 134 (e.g., running/runs/runner→run; better→good), which groups related variants and improves model efficiency. During word embedding 126, the normalized tokens are converted into numerical vectors 138 in a high-dimensional space that encodes each word's contextual relations and sentiment associations; these vectors can be priority-weighted for specialty codebook scoring and fused with other features. Where applicable, contextualization and processing 140 (e.g., transformer/LSTM sequence modeling) are applied to capture long-range dependencies and utterance-level meaning. The system may additionally employ TextBlob to extract polarity and subjectivity scores that feed into the real-time analytics pipeline.

For stop words removal in Python, an example code sequence is:

Python
Copy
import nltk
from nltk.corpus import stopwords
from nltk.tokenize import word_tokenize

For lemmatization and stemming, an example code sequence is:

python
Copy
from nltk.stem import WordNetLemmatizer, PorterStemmer

For word embedding, an example code sequence is:

python
Copy
from textblob import TextBlob
text = TextBlob(example_sentence)
text.tags
text_sentiment = text.sentiment

For contextualization and processing, an example code sequence is:

```
python
Copy
def analyze_sentiment(text):
  # Implementation details
  pass
sentiments = [analyze_sentiment(sentence)
```

FIG. 1B provides preferred steps to develop simulated patients/agents in of FIG. 1A. Patient histories are received 150. This can include patient histories, case histories regrading a disease for which a patient was treated. This is available through on-line resources such as the Mayo Clinic and provides medical data on disease.

An AI source is trained 152 with personality traits. Open AI GPT is trained, for example, with the 152 Human Personality Factors and a motivational considerations document. Other open sources or commercial platforms can be trained in the same way.

A de-identified white sheet (the prompt code) 154—combining (i) case-specific behavior prompts, (ii) the historical/contextual setting of the encounter, and (iii) the medical facts of the case—is provided to the LLM to generate a character profile sheet that specifies the patient persona, disclosure rules/guardrails 120, and symptom/response seeds aligned to the scenario. The character profile sheet is then submitted to a checking/review application (e.g., the Claude AI application) to check for contradictions, inconsistencies, and unintended disclosures, and to confirm alignment with the applicable specialty codebook 124 (e.g., trust-building and teach-back cues for Internal Medicine). Profiles that fail any check are flagged with corrective annotations and re-run; approved profiles are versioned under a case ID for reproducibility and passed downstream to generate a detailed physical description 156, optional visual/voice assets 158/164, and to initialize response policies for the voice-to-voice simulation via the steps 126-140.

The same GPT is then used to generate 156 a detailed physical description of the character. An image generating agent uses the physical description to generate an accurate image of the character. An example image generator is the Leonardo AI image generator.

A visual agent is then created 158. D-iD can be used to create a visual agent. Instructions are inputted to a site for a visual agent and a photo from the image generator is provided. Training is conducted with the character profile information.

Agent responses are then created 160. Open AI GPT 4o can be used as the LLM that generates agent responses. An alternative is to use a local model to generate character responses.

The character profile can then be edited 162. The character profile information is used and leading behaviors can be changed or additional facets added. This can be done with human input or a creative writing centric LLM can conduct it automatically.

Agent voice modification 164 can be conducted. Elevenlabs software or Speechify AI can be used for voice modification.

A 3D model can be created 166 as part of agent creation. Nvidia Digital humans or Unreal Metahumans can be used for the 3D agent creation.

FIGS. 1C-1D provides preferred steps for sentiment analysis for patient simulations. Codewords are received from a medical school and a codebook is created 166. The codebook is a collection of text phrases used in medical practice. The phrases consists of codewords that are focused on specific diseases and essential for effective communications between a professional and a patient in a medical setting.

During a live encounter with a trainee, the software executes the entire pipeline in real time: it captures voice-to-voice communications 126, performs ASR with tokenization/stop-word removal 130, lemmatization and stemming 132, and word embedding 136, then applies contextualization and processing 140 to attribute each utterance to the correct speaker and derive features (e.g., sentiment strength, intent, latency/hesitancy). Detected terms are matched on-the-fly against the specialty language codebook 124. The system then converts and prioritizes codewords 168 by extracting them from the codebook and assigning a dynamic priority weight based on (i) sentiment polarity/strength and (ii) importance to the current clinical task and learning objectives. These priority weights update turn-by-turn as context evolves; hits/misses and priorities feed the measurement layer and the real-time interview display 144, while guardrails 120 enforce disclosure rules.

Codewords are then converted 170 to text/vectors values. Vector quantization can be conducted to convert codewords into a dictionary or reference for mapping input data to discrete values. Transformer models such as GPT used codebooks in this matter to reduce memory and computational costs.

Voice is converted to text 172. Spoken audio is converted to text using a tool, such as Google speech to text. Text can be extracted, for example using Whisper AI and other Python libraries. Conducting voice recognition followed by text extraction allows speakers to be identified using diarization. Audio/speech→Preprocessing→Speech Recognition→Text output. Getting text output from D-ID can reduce the cost/need of the text extraction process.

Software analysis 174 can use Python language and tools NLTK And SPaCy. Traditional Python libraries can also be used, such as text blob, to calculate polarity indexes. [0062] In the pipeline of FIG. 1C, software analysis 174 is implemented in Python using NLTK and spaCy for tokenization, lemmatization, and part-of-speech tagging; additional libraries such as TextBlob compute sentiment polarity and subjectivity indices that feed the real-time scoring and reporting layers.

Text is tokenized 176 into individual word tokens; these tokens are then used for analyzing sentiment in SA (Sentiment Analysis). In this specification, SA means Sentiment Analysis (not situation awareness). Tokenization is performed with Python's NLTK (or spaCy), producing tokens that feed subsequent lemmatization/stemming 132, word embedding 136, and contextualization and processing 140 steps.

Lemmatization and stemming 178 can be conducted by software. This provides text normalization in an NLP tool (natural language processing) that reduces words to their base or root forms. This is a function in the NLTK tool.

Conceptualization processing 182 is conducted via a model. The model semantic provides understanding, concept extraction and knowledge representation.

Word embedding 180 is conducted via NLP software that assigns tokens priority ranks. The embedding captures contextual essence include sentiment relations between works. Numerous APIs can be used to provide this function in tensorflow or Open AI platforms.

Speaker diarization 184 distinguishes between the trainee speaker and the agent. Audio is divided into segments according to recognized voices. Combined with text analysis, this provides AI software with the ability to understand a question and answer session during the simulation.

Context is interpreted 186a. This can be conducted by using long-short-term-memory (LSTM) network transformation to build an LLM.

Storage and flow 186b are conducted. This can be done with the LSTM, which can store information that has gates for regular flow of information.

Gate details are constructed 186c. A cell state carries information across a sequence and remembers important context over long sequences. A forget gate decides information from a previous cell that can be discarded. An input gate decides information to store in a cell state. An output gate controls the information sent to the next layer used in a current output state.

Long term dependencies are captured 186d. Context in the SA is important for long term dependencies to understand text sentiment. Sentiment prediction allows a hidden state in the LSTM to help understand the text and predict sentiment using neural network activation layers, e.g. Softmax and Sigmoid.

The LSTM network is trained 186e. This uses the specialty language codebooks that trains the local language model (LLM) so it is tailored for disease-specific interactions, ensuring accurate learning metrics during a simulation.

The LSTM is used in sentiment analysis 186f. This provides context awareness (remembering important words), handling long texts and capturing sequential dependences (a highest priority word can, for example, change an entire sentiment and LSTM understands differences that change sentiment). This can be optimized with a stochastic gradient descent or via the adam ai platform.

Coding for providing an AI patient display is described next. The preferred coding includes the following steps.
1. Set Up Your Environment
   Python≥3.10
   Install: opencv-python, pyaudio (or sounddevice), ffmpeg (system), numpy, transformers (or your ASR/LLM client), nltk, spacy, textblob, matplotlib (for the timeline), and a small web/GUI layer (e.g., streamlit or simple OpenCV windows).
   Download spaCy model (e.g., en_core_web_sm) and NLTK corpora if your pipeline needs them.
2. Prepare case inputs (the “white sheet”)
   Load the de-identified case JSON/YAML containing:
      Persona & dialog seeds (prompt code)
      Encounter context (e.g., clinic vs ED)
      Medical facts & disclosure guardrails
   Select the internal-medicine (or relevant specialty) codebook.
3. Initialize display controller (“Smartphone” frame)
   Create a phone-sized canvas (e.g., 1080×1920 or proportional) with three regions:
      404: patient video/agent viewport
      406: Codebook Score Panel (live hit/miss badges)
      408: Sentiment/State Timeline (rolling graph)
4. Start capture & audio
   Open the agent video stream (file, camera, or avatar output).
   Start the learner mic capture (mono or 2-ch).
   Set timestamps from a single master clock.
5. Enable speech-to-text (ASR)
   Route mic audio to your ASR (e.g., Whisper/Cloud ASR).
   Request word-level timestamps to align text with frames.
6. Run the NLP preprocessing
   Tokenization/stop-word removal (130)→filter to content words.
   Lemmatization/stemming (132/134)→normalize variants.
   Word embeddings (136/138)→convert tokens to vectors.
   Optional: TextBlob polarity/subjectivity; spaCy POS if needed.
7. Compute real-time features
   Latency/hesitancy (ask→answer gap), speaking turns, diarization.
   Sentiment/rapport (rolling windows).
   Codebook matches (hit/miss, priority weighting).
8. Apply guardrails
   Enforce disclosure rules (e.g., patient cannot self-diagnose unless unlocked).
   Redirect agent outputs if a rule would be violated.
9. Render the UI each tick
   404: draw the current video frame (or avatar) and speaking indicator.
   406: update codeword list with color badges and counts.
   408: append latest metrics to the strip-chart (valence, arousal, rapport); show markers for questions, disclosures, escalations.
10. Interact & monitor
   Ask the patient questions; observe live updates in 406/408.
   Use on-screen controls to pause, scrub, or switch Simulation/Arcade mode.
11. Log and export
   Save synchronized logs (audio, transcript with timestamps, hits/misses, sentiment timeline).
   Generate a session report PDF/CSV and (optionally) push via API to your LMS/assessment system.
12. Troubleshooting tips
   Blank video→verify the video source/URL and OpenCV codec.
   No ASR text→check mic device index, sample rate, and API key/rate limits.
   Frozen timeline→ensure your render loop runs on a separate thread from ASR/NLP.
   Misaligned captions→confirm all modules use the same master clock.

Coding for providing a smartphone application of the present medical patient simulation training system is described next. The preferred coding includes the following steps.
1. Prep your environment
   Python 3.10+
   Install:
      pip install opencv-python numpy pyaudio sounddevice transformers torch nltk spacy textblob matplotlib
   (Optional) Models/data: python-m spacy download en_core_web_sm, NLTK corpora if needed.
2. Load the case (“white sheet”) and codebook
   Read your white_sheet.json (prompt code+setting+medical facts).
   Read your codebook.json for the chosen specialty (e.g., Internal Medicine).
3. Build the “smartphone” UI canvas
   Create a tall window (e.g., 1080×1920 or scaled).
   Reserve three regions:
      404: patient video/agent viewport (top).
      406: Codebook Score Panel (left or right strip).
      408: Sentiment/State Timeline (bottom strip chart).
4. Initialize capture & master clock (Module 1)
   Open patient video stream (file, webcam, or avatar feed).
   Open mic input (mono or 2-ch).
   Start a master timestamp so all modules stay in sync.
5. Start ASR for voice-to-voice (126→text)
   Stream mic audio to ASR (e.g., Whisper or cloud ASR) with word-level timestamps.
   Buffer utterances with start-end times for alignment.
6. Run NLP preprocessing in real time
   Tokenization & stop-word removal (130) on each utterance.
   Lemmatization/stemming (132/134) to normalize word forms.
   Word embeddings (136/138) to turn words into vectors.
   (Optional) TextBlob polarity/subjectivity; spaCy POS tags.
7. Contextualize sequences (140)
   Use a Transformer/LSTM to model turn-by-turn context, long-range cues, and diarization (who spoke).
8. Compute live metrics
   Latency/hesitancy (question→answer delay).
   Sentiment/rapport rolling scores.
   Turn taking, disclosure events, escalation markers.
9. Match the specialty codebook (124)
   Detect codeword hits/misses per utterance.
   Prioritize (168) by importance and sentiment strength.
   Apply guardrails (120) to prevent forbidden/premature disclosures.
10. Render each frame (the display you see)
   404: draw patient video+speaking indicator.
   406: update the Codebook Score Panel (colored badges, counts, priorities; tap/hover shows examples).
   408: append to the Sentiment/State Timeline (valence/arousal/rapport vs time, with event markers).
11. Provide real-time feedback (144)
   Surface turn-by-turn tips (e.g., “Try a teach-back question”).
   Let the user toggle Simulation vs Arcade mode if implemented.
12. Log and export
   Save synchronized: audio, transcript (with timestamps), codebook hits/misses, timeline arrays.
   Generate a PDF/CSV session report; optionally POST to your LMS/assessment API.
13. Clean up
14. Quick troubleshooting steps
   Release camera/mic, close windows, flush logs.
   No video→check stream path/codec and OpenCV build.
   No transcript→confirm mic device index/sample rate; verify ASR keys/limits.
   Laggy UI→run ASR/NLP in a worker thread; keep the render loop light.
   Misaligned captions→ensure all modules reference the same master clock.

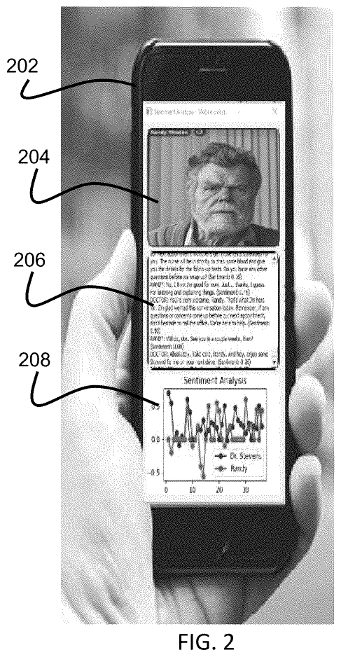

FIG. 2 shows a mobile device 202 with an example display during a simulated patient interaction. A live image/video simulation 204 of the patient is on a portion of the screen. The mobile device 202 shows the following in FIG. 2.

### 204—Patient Viewport (Live Image/Video)

The left pane shows the AI-simulated patient's face (video/avatar). A speaking indicator would appear here during the learner's or patient's turn. This pane is driven by the case's white sheet (prompt code) with guardrails 120 controlling what the patient can/can't disclose, and it reflects the active encounter context (clinic, ED, telehealth).

### 206—Codebook Score Panel

The middle pane is a text/metrics panel that updates in real time as the conversation is analyzed. It lists specialty codebook 124 items (e.g., trust-building prompts, teach-back checks) and shows hit/miss status, priorities, and counts. This is powered by the live NLP pipeline (ASR 126,Üi tokenization/stop-word removal 130,Üi lemmatization/stemming 132/134,Üi embeddings 136/138,Üi contextualization 140) and the measurement layer 144.

### 208—Sentiment/State Timeline

The right pane is a scrolling chart of interaction state over time (e.g., valence, arousal, rapport) with markers for key events-questions, disclosures, escalations, codebook hits. It visualizes the fused output of verbal+nonverbal analysis and updates continuously during the session.

In short: the screen is the runtime UI for the AI patient simulation-patient video on the left (204), live codebook scoring in the center (206), and the rolling sentiment/state graph on the right (208)-all running on the device (202) and updated in real time by the software pipeline.

When a trainee is using the app, the screen is divided into three live panels that update continuously during the encounter:

Patient viewport 204 (left): a live video/avatar of the AI-simulated patient that speaks and reacts according to the case's white sheet (prompt code) and guardrails 120. Tone and phrasing adapt to the encounter setting (clinic, ED, telehealth).

Codebook Score Panel 206 (center): a real-time checklist derived from the specialty codebook 124 (e.g., trust-building questions, teach-back, risk explanation). As the learner speaks, the software performs ASR and NLP (tokenization/stop-words 130, lemmatization/stemming 132/134, embeddings 136/138, contextualization 140) and immediately marks hits/misses with priority weights. This panel provides turn-by-turn guidance on which required elements were covered or are still missing.

Sentiment/State Timeline 208 (right): a scrolling graph of fused interaction metrics (e.g., valence, arousal, rapport) with event markers for key questions, disclosures, and escalations. The timeline reflects measurements computed in real time and surfaced via the interview analysis and display 144.

In operation: when the learner asks an empathy or teach-back question, 206 registers a codebook hit and 208 often trends upward for rapport; if a guardrail would be violated (e.g., premature self-diagnosis), the system redirects the patient response and no disclosure marker appears. Optional dual-camera input can show a picture-in-

The app run on the mobile device 202 first uploads the case ‘white sheet’—the prompt code that defines the patient's behavior, the clinical setting, and the medical facts.

The phone-style display then opens with three areas: the patient video 204, the codebook score panel on the side (206), and the sentiment timeline along the bottom (208).

When the session starts, the system listens to the conversation. It turns speech into text, breaks the text into words, normalizes them, and creates vectors so the software understands meaning and context. In real time, it measures things like who's speaking, how long responses take, and the emotional tone of the interaction.

As the learner asks questions, the software checks for specialty codebook phrases—trust-building prompts, teach-back checks, and other required elements. Hits and misses appear instantly in the score panel. Guardrails prevent the virtual patient from revealing information before it's appropriate for the case.

The timeline updates continuously with sentiment and rapport, and events like key questions or disclosures are marked as they happen. At the end, the system exports a synced report—audio, transcript, codebook scores, and the timeline—so instructors and learners can review performance and next steps.”

One mode is a Simulation Mode (High-Fidelity): In this state, the system operates like prior deterministic systems. The AI patient's responses are governed by a strict, evidence-based algorithm, ensuring the scenario is a predictable and repeatable training exercise for medical professionals.

Another mode is Arcade Mode (Low-Fidelity): As the user moves a GUI switch, the system begins to introduce a calibrated degree of non-determinism. It intentionally deviates from the optimal, ‘textbook’ path. The AI patient generates responses that are still clinically plausible but are deliberately unanticipated by the learner. The system expands a predictable training tool to be able to provide an unpredictable and engaging game of medical patient simulation, which game is constructed from models trained with specialty codebooks as discussed above.

The system provides a dual-purpose platform from a single simulation engine. This transforms a niche professional training tool into a gamified experience with training benefits that provide a real-world experience. The arcade mode provides teaching adaptability and critical thinking, rather than just procedural steps. It creates an environment where the outcome is not a foregone conclusion, thereby advancing simulation training.

Overall flow of training of a preferred embodiment medical patient simulation training system used on a desktop system or the mobile app of FIG. 2 can proceed as follows. Collect real examples
Gather transcripts, standardized-patient scripts, clinical guidelines, and teaching checklists from the target specialty (e.g., Internal Medicine, ED).
Extract a specialty codebook
Pull out the phrases, question stems, and responses that matter (e.g., empathy statements, red-flag questions, teach-back prompts). Normalize wording and define synonyms.
Prioritize the codewords
Assign weights based on clinical importance and learning objectives (e.g., safety questions>rapport builders) and note their typical sentiment/intent.
Preprocess the data
Clean text, tokenize, remove stop-words, lemmatize/stem; align each utterance to a speaker and timestamp (diarization).
Convert words to vectors
Build embeddings so the model understands meaning and relationships among terms (context, sentiment, and proximity to codewords).
Train the recognition models
Use sequence models (Transformer/LSTM) to detect required phrases, intent, and sentiment across turns-not just isolated keywords.
Fine-tune & calibrate
Adjust thresholds to reduce false positives/negatives, test across scenarios, and lock in “what counts” as a valid hit for each codeword.
Author the case (“white sheet”)
Combine prompt code+context+medical facts; set guardrails so the AI patient follows the script and doesn't reveal information prematurely.
Run the live session
Capture voice-to-voice conversation; perform ASR; process text in real time; detect codebook hits/misses; compute sentiment/rapport and latency.
Display live feedback
Patient video (viewport)
Codebook Score Panel showing hits/misses and priorities
Sentiment/State Timeline with event markers
Score trust-building & comprehension
Aggregate detected behaviors (e.g., empathy, teach-back success) into interpretable “rulers” that track rapport and understanding.
Report & improve

Export synchronized logs and a session report; update codebook weights and model parameters based on outcomes-closing the learning loop. “We start by collecting real clinical language from transcripts, SP scripts, and guidelines. From that, we build a specialty codebook—a curated list of the phrases and questions we want learners to use. We rank those items by importance, then clean and prepare the data so the software can understand who's speaking and what's being said.

Next, we convert words into vectors and train a sequence model to recognize these behaviors in context, not just as isolated keywords. We fine-tune the model and thresholds so detections are accurate.

With that in place, we author each case as a ‘white sheet’ that sets the patient's behavior, the setting, and the medical facts, and we add guardrails to prevent premature disclosures. During a session, the system listens to the voice-to-voice dialog, turns it into text, and—moment by moment—checks for codebook items, measures sentiment and rapport, and tracks timing.

On the screen, you see the patient video, a codebook score panel that lights up hits and misses, and a timeline of the interaction state. At the end, everything is saved into a report, and we use those results to refine both the codebook and the model for the next run, steadily improving trust-building and clinical communication.”

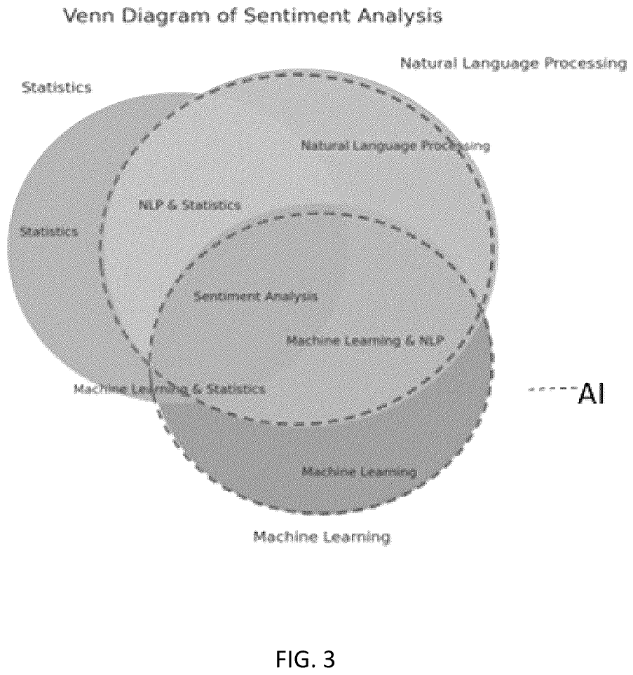

FIG. 3 shows a Venn diagram of the sentiment analysis used in preferred embodiments. Natural Language Processing (NLP)—
Turns raw language into analyzable structure.
Tasks: tokenization, stop-words, lemmatization/stemming, POS tagging, phrase/chunk detection, embeddings.

### Statistics—

Quantifies patterns and uncertainty.
Tasks: frequency counts, co-occurrence, correlation, hypothesis tests, confidence intervals, calibration curves.

### Machine Learning (ML)

Learns predictive patterns from data.
Tasks: classifiers/regressors (logistic regression, SVM), sequence models (LSTM/Transformers), evaluation (ROC/AUC, F1).
Overlaps (where the circles intersect)
NLP & Statistics: lexicon scores, term weighting (TF-IDF), sentiment lexicons, n-gram probabilities.
ML & S Statistics: feature selection/regularization, error analysis, model calibration, uncertainty estimation.
ML & S NLP: learned representations (Word2Vec/GloVe/Transformer embeddings), sequence modeling of text, intent classification.
Center (all three overlap): Sentiment Analysis
Combines NLP (to structure language)+Statistics (to measure)+ML (to predict).
Outputs: polarity (positive, negative), intensity, subjectivity, and higher-order states (e.g., rapport).
These feed the real-time dashboard (codebook hits, sentiment/rapport timeline).
Dashed boundary labeled “AI”

Highlights that applied AI in this context is largely the ML+NLP region plus statistical grounding-to make decisions and feedback in real time. Think of sentiment analysis as the place where three skill sets meet.
First, NLP turns messy speech or text into structured tokens and meanings.
Second, Statistics measures what we see—how often terms appear, how strongly they relate, and how confident we are.
Third, Machine Learning learns patterns from those measurements to predict whether something is positive, negative, or neutral, and how strong that feeling is.

Where all three overlap is sentiment analysis. That's what the system uses to score each turn in the conversation: NLP structures the words, statistics quantifies the signals, and machine learning makes the real-time judgment. The dashed boundary shows the AI layer that wraps this all together so the app can provide live feedback—like rapport trends and codebook hits—while the encounter is happening.

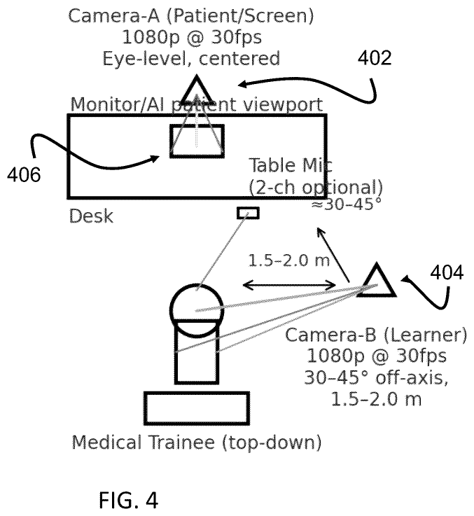

FIG. 4 shows a preferred system that includes a camera A 402 and a camera B 404 that are positioned to capture both a trainee and a virtual patient shown on a monitor 406. The following description is of software of a preferred medical patient simulation training system including the c. The system can capture, analyze, and interpret the nonverbal “inner voice” communications between a medical learner and an AI-simulated patient during training scenarios. The system can provide objective, data-driven feedback on the learner's interpersonal skills by measuring subtle, nonverbal cues that indicate the underlying emotional and cognitive states of both participants.

### Module 1: Data Capture Layer

This layer is configured to capture high-fidelity, synchronized audio-visual data and text interactions from the simulation environment.

### 1 Video Capture

Hardware: Implement support for two high-definition (1080p, 30 fps minimum) video cameras.
Camera-A 402 (Patient): Focused tightly on the AI-simulated patient's face and upper torso.
Camera-B 404 (Learner): Focused on the medical learner's face and upper torso.

Software: Capture module using a multi-camera API (e.g., OpenCV, PyAV) capable of simultaneously accessing and recording from both video streams. The module must handle device enumeration and selection.

### 2 Audio Capture

Hardware: 2-channel microphone to capture distinct audio from the patient and the learner.

Software: Records audio in a lossless format (e.g., WAV or FLAC). Each channel processed independently.

### 3 Text I/O

Interface: UI captures text-based prompts or chat messages exchanged between the learner and the simulated agent.

Data Structure: Logs text interactions with precise timestamps to align with the audio/video streams. Provides supplementary data source for contextual analysis.

### Module 2: Preprocessing and Synchronization

This module aligns data streams and converts raw date into a usable format for analysis.

### 1 System Check & Timestamping

Upon session initiation, the system will perform a hardware check to ensure all cameras and microphones are operational.

A master clock is established. All incoming data packets (video frames, audio chunks, text logs) will be timestamped against this master clock to ensure sub-second accuracy.

### 2 Multi-Stream Synchronization

Synchronization algorithm aligns video frames and audio chunks based on their timestamps. Allows accurate multimodal analysis. Audio diarization separates and labels speech segments from the learner and the patient/agent.

### 3 Speech-to-Text (ASR)

Automatic Speech Recognition (ASR) engine (e.g., Google Cloud Speech-to-Text, Whisper) transcribes audio from both channels.

The ASR output must include word-level timestamps to align the spoken content with nonverbal cues.

### 4 Face Detection & Landmarks (Interim Step)

For each video frame, a facial detection library (e.g., Dlib, MediaPipe) identifies the face region and extract key facial landmarks (e.g., corners of eyes, mouth, nose tip). This data is used for facial expression analysis.

### Module 3: Nonverbal Feature Vector Extraction

This module converts raw visual data into quantitative feature vectors representing nonverbal behaviors.

### 1 Pose Skeleton Extraction

Pose estimation model (e.g., OpenPose, MediaPipe Pose) extracts a 2D or 3D skeletal model from each video frame for both the patient and the learner. This tracks body language and posture.

### 2 Consent/Privacy Filters

Filtering module can blur faces or abstract visual data into stick figures to protect the identity of the learner, if required by privacy protocols.

### 3 Facial Action Units (FAUs) & Micro-Expressions

Extracted facial landmarks are analyzed and classified FAUs based on the Facial Action Coding System (FACS). This FACS measures muscle movements associated with specific emotions.

Model sensitivity is set high enough to detect micro-expressions (brief, involuntary expressions).

### 4 Posture, Orientation & Fidget Indices

The pose skeleton date is used to calculate metrics.
Posture: Leaning forward (engagement) vs. leaning back (disengagement).
Orientation: Body and head angle relative to the other person.
Fidget Index: Quantify repetitive or nervous movements (e.g., hand-wringing, leg shaking).

### 5 Gaze & Attention Cues

Gaze tracking uses facial landmarks to estimate the direction of eye gaze. This helps determine focus and attention.

### 6 Stick & Figure Icon Generator

Rendering module visualizes the nonverbal data.
Pose Skeletons: Renders the extracted skeletons as real-time stick figures.
Facial Glyphs: Map the detected FAUs and micro-expressions to a set of standardized glyphs or icons that visually represent the detected emotion (e.g., a frowning icon for negative sentiment).

### Module 4 Channel Separation

Isolates and analyzes different communication channels independently before fusion. Uses the following date streams.
Latency/Hesitancy Metrics Calculate the time delay between a question being asked and an answer beginning. Analyze speech patterns for hesitations (e.g., filler words, pauses)
Keystroke Dynamics (If Applicable) If text input is used, analyzes typing speed, error rate, and rhythm as potential indicators of stress or uncertainty.
Verbal Channel (Context Only) Timestamped ASR transcript serves as the verbal channel. It is used primarily to provide context for the nonverbal analysis, not for sentiment analysis itself.
Inner Dialogue Stream (Nonverbal). Composite stream created by combining all extracted nonverbal feature vectors (pose, facial cues, gaze, etc.) into a single, time-series data structure.

### Module 5 Codebook & Fusion Engine

Interprets the nonverbal data stream with a framework and fuses the channels into a coherent analysis.

### 1 Codebook

Rule-based dictionary (Codebook) that maps specific nonverbal cues and combinations of cues to affective states (positive, negative, neutral). Example Rule: IF (FAU-12 ‘lip corner pull’ is active) AND (Posture is ‘leaning forward’) THEN (Inner Voice State=Positive Engagement)

### 2 Multimodal Fusion Engine

Fusion engine that processes the Inner Dialogue Stream through the Codebook

Engine uses a combination of rules and a trained machine learning model (e.g., a Recurrent Neural Network—RNN) to interpret the sequence of nonverbal cues over time.

3 Patient vs. Learner State Tracks

The engine maintains separate, parallel state tracks for the patient and the learner, allowing for analysis of interaction dynamics (e.g., emotional contagion, rapport).

### Module 6 Ruler Scoring Engine (Dimensions)

Score interactions along predefined psychological dimensions based on the analyzed inner dialogue.

The Scoring Pipeline includes:
Inner Dialogue Analysis: The pipeline takes the output from the Fusion Engine as its input.
Dimensional Scoring: The engine applies a set of “rulers” to score the interaction on various dimensions. Each ruler is a computational model that quantifies a specific trait. Many traits can be used, including the following examples.
   Sentiment: Positive vs. Negative emotional valence.
   Fear/Anxiety: Cues related to stress and nervousness.
   Anger/Frustration: Cues related to irritation or conflict.
   Trust/Rapport: Cues indicating comfort, engagement, and connection.

### Module 7 Output and Storage

Presents the analysis in an accessible format and securely store the data.
Real-Time Dashboard. A web-based UI that displays the dimensional scores, rendered stick figures, and facial glyphs in real-time during the simulation. This provides immediate feedback.
Session Report & Export. Post-session, generates a comprehensive PDF report detailing the scores over time, highlighting key events, and providing actionable feedback.
Anonymized Data Source. All collected data (feature vectors, scores, anonymized transcripts) is stored in a secure database. This data lake provides support for future research and model refinement.
API for Assessment. API that allows external learning management or assessment systems to retrieve session scores and reports for a given learner, enabling seamless integration into existing educational workflows.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A medical patient simulation training system, comprising:
a display interface for a trainee using the training system;
a trained artificial intelligence agent, wherein the agent is trained on a codebook for disease-specific interactions; and
real-time software that quantifies the use of codebook words and phrases, assesses diagnostic accuracy and emotional engagement of the trainee during a simulation training session.

2. The system of claim 1, wherein the artificial intelligence agent is trained with personality traits that affect patient-professional interactions in a medical setting.

3. The system of claim 2, wherein the artificial intelligence agent is trained on the codebook words and phrases and the codebook words and phrase are provided locally by a medical education department.

4. The system of claim 2, wherein the artificial intelligence agent is trained for voice-to-voice communications and facial expressions.

5. The system of claim 2, wherein the artificial intelligence agent is trained with motivational considerations.

6. The system of claim 2, comprising a) a data acquisition module for obtaining medical information; b) a natural language processing engine for generating a plurality of patient profiles to serve as the artificial intelligence agent; c) an image generation module for creating visual representations of the artificial intelligence agent; d) an AI personality model for integrating profile and visual data of the artificial intelligence agent; e) a testing and refinement module for refining the artificial intelligence agent; and f) a deployment module for presenting the artificial intelligence agent to the trainee.

7. The system of claim 2, wherein the artificial intelligence agent is generated using a natural language processing engine that utilizes a fine-tuned Generative Pre-trained Transformer (GPT) model.

8. The system of claim 2, wherein the artificial intelligence agent is generated using an image generation module that includes a Stable Diffusion XL (SDXL) model.

9. The system of claim 2, wherein the artificial intelligence agent is trained with medical data from accredited sources.

10. The system of claim 9, wherein the artificial intelligence agent updated based upon trainee feedback and updated medical data.

11. The system of claim 2, comprising an output that includes a sentiment analysis report generated by analysis of trainee-artificial intelligence interaction.

12. The system of claim 11, wherein the sentiment analysis report includes identification of emotions including happiness, sadness, anger, pain, frustration, empathy, pity, and excitement.

13. The system of claim 12, wherein the sentiment analysis report includes an assessment of the urgency of the situation and patient intentions.

14. A medical patient simulation training system:
an autonomous AI platform featuring visually simulated patients trained to provide voice-to-voice communication with natural intonations and to interact and respond to a trainee;
a local language model trained on specialty language codebooks tailored for disease-specific a simulation interaction with a trainee; and
real-time software that quantifies the use of codebook words and phrases by the trainee, and assesses diagnostic accuracy and emotional engagement of the trainee during the simulation interaction.

15. The system of claim 14, comprising an output interface for the user, wherein the output interface allows the trainee to select simulation mode that provides deterministic responses from the visually simulated patients and an arcade mode that provides non-deterministic responses.

16. The system of claim 15, wherein the deterministic responses are governed by a strict, evidence-based algorithm, ensuring the scenario is a predictable and repeatable training exercise for medical professionals and the non-deterministic responses are clinically plausible but deviate from the evidence-based algorithm.

17. The system of claim 15, comprising a first camera configured to capture the visually simulated patients and a second camera configured to capture the trainee.

18. The system of claim 17, comprising an interface that captures one or more of voice interactions, text-based prompts or chat messages exchanged between the learner and the visually simulated patients.

19. The system of claim 17, comprising processing to extract pose, posture, orientation and/or fidget information regarding the learner and the visually simulated patients.
